(19) United States
(12) Patent Application Publication
(10) Pub. No.: US 2015/0051139 A1
(43) Pub. Date: Feb. 19, 2015

(54) Mucins as Antiviral Compounds

(71) Applicant: Massachusetts Institute of Technology, Cambridge, MA (US)

(72) Inventors: Oliver Lieleg, Garching (DE); Katharina Ribbeck, Cambridge, MA (US)

(73) Assignee: Massachusetts Institute of Technology, Cambridge, MA (US)

(21) Appl. No.: 14/376,656

(22) PCT Filed: Feb. 6, 2013
(86) PCT No.: PCT/US13/24936

§ 371 Date: Aug. 5, 2014

Related U.S. Application Data
(60) Provisional application No. 61/595,649, filed on Feb. 6, 2012.

(51) Int. Cl. A61K 38/17; G01N 33/50; A61K 9/00

(52) U.S. Cl. CPC A61K 38/1735; A61K 9/0014; G01N 33/5091
USPC 514/3.7; 435/375; 435/5

(57) ABSTRACT

The invention provides methods, and compositions for performing the methods, that reduce the diffusion or overall mobility of a virus on a surface, such as a biological surface using a purified mucin. The methods can reduce the infectivity of a virus for a cell on the surface. In particular embodiments, the mucin can be a non-human mucin, such as a procine gastric mucin.

### RELATED APPLICATION

This application claims the benefit of U.S. Provisional Application No. 61/595,649, filed on Feb. 6, 2012.

The entire teachings of the above application are incorporated herein by reference.

### GOVERNMENT SUPPORT

This invention was made with government support under P50-GM068763 and P30-ES002109 from the National Institute of Health. The government has certain rights in the invention.

### BACKGROUND

Viruses remain a scourge to human health and well being. Vaccines can be effective, but high mutation rates of viruses, the wide variety of viruses, and the high cost of vaccines limit their overall effectiveness. Accordingly, a need exists for additional methods of inhibiting viral infection (e.g. by limiting viral diffusion in the extracellular space), preferably the methods would show broad-spectrum antiviral effectiveness, and further still, the methods should be low-cost.

### SUMMARY OF THE INVENTION

The invention provides, inter alia, low cost, methods that show broad-spectrum activity for limiting viral diffusion on a surface and/or that can reduce viral infectivity.

In a first aspect, the invention provide a method of inhibiting infection of one or more cells by a virus comprising contacting the one or more cells with a composition comprising biocompatible, purified, mucin. In certain embodiments, the mucin is a gastric mucin and in more particular embodiments, the gastric mucin is a non-human gastric mucin, such as a porcine gastric mucin. In more particular embodiments, the composition comprises principally MUC5AC. While the virus inhibited by these methods may be any virus, in certain embodiments, the virus is not a norovirus.

In some embodiments, the mucin is a non-recombinant purified from a biological source. In other embodiments, the mucn is a recombinant mucin.

In certain embodiments, the concentration of the mucin is lower than the mucin concentration of wild type mucin, e.g. in vivo. In particular embodiments, the concentration of the mucin is about 4- to about 5-fold lower than the mucin concentration in vivo.

In particular embodiments, wherein the mucin concentration is about 0.20% (w/v), 0.25% (w/v), 0.30% (w/v), 0.35% (w/v), 0.40% (w/v), 0.45% (w/v), 0.50% (w/v), 0.55% (w/v), 0.6% (w/v), 0.65% (w/v), 0.7% (w/v), 0.75% (w/v), 0.8% (w/v), 0.85% (w/v), 0.9% (w/v), 0.95% (w/v), 1% (w/v), 1.5% (w/v), 2.0% (w/v), 2.5% (w/v) when hydrated. In more particular embodiments, the mucin concentration is between about 0.125 to about 2.0% (W/V). In still more particular embodiments, the mucin concentration is between about 0.2 to about 1.2% (W/V).

The pH of the mucin-containing compositions can be adapted to pH suitable for any mucin used consonant with the invention. For example, in some embodiments, the composition has an acidic or neutral pH. In more particular embodiments, the composition has an acidic pH. In certain embodiments, the pH is about 2, 3, 4, 5, 6 or 7. In more particular embodiments, the composition has a pH of about 2 to about 4.

In certain embodiments the composition has a salt concentration with an ionic strength equivalent to a 1:1 electrolyte of about 20 nM, 40 mM, 60 mM, 80 mM, 100 mM, 120 mM, 140 mM, 160 mM, 180 mM, 200 mM, 220 mM, 240 mM, 260 mM, 280 mM, 300 mM, 320 mM, 340 mM, 360 mM, 380 mM, 400 mM, 420 mM, 440 mM, 460 mM, 480 mM, 500 mM, 520 mM, 540 mM, 560 mM, 580 mM, or 600 mM when hydrated.

In some embodiments, the virus has a diameter of about 30 nm, 40 nm, 50 nm, 60 nm, 70 nm, 80 nm, 90 nm, 100 nm, 120 nm, 140 nm, 160 nm, 180 nm, or 200 nm—e.g. non-noroviruses can be in these size ranges. Exemplary viruses targeted by the methods provided by the invention include a human papilloma virus type 16 (HPV 16), a Merkel cell polyoma-virus (MCV), an influenza virus, human immunodeficiency virus (HIV), Herpes simplex virus (HSV), Hepatitis B, Hepatitis C or a combination thereof. In more particular embodiments, the virus is a human papilloma virus type 16 (HPV 16), a Merkel cell polyoma-virus (MCV), an influenza virus, or a human immunodeficiency virus (HIV). In still more particular embodiments, the virus is a human papilloma virus type 16 (HPV 16), a Merkel cell polyoma-virus (MCV), or an influenza A virus.

Compositions for use in the methods provided by the invention can be in a variety of forms, including both hydrated and lyophilized forms. In certain embodiments, the composition is provided in a cream, lotion, ointment, or aerosolizable form suitable for external or internal use by a human. In some embodiments, the composition is provided in a bandage form, e.g. optionally wherein the composition is lyophilized.

In some embodiments, the surface to be treated by the methods provided by the invention comprises animal cells, preferably mammalian cells, more preferably human cells. In certain embodiments, the cells are cultured. In other embodiments, the cells are in vivo, in a subject, e.g. a human subject.

The compositions provided by the invention may, in certain embodiments, reduce the rate of diffusion or movement more generally, such that the virus exhibits a mean square displacement per second of less than 2.0 μm² on a surface. In more particular embodiments, a virus exhibits a mean square displacement per second of less than 0.5 μm² on a surface. In still more particular embodiments, a virus exhibits a mean square displacement per second of less than 0.25 μm² on a surface.

In another aspect, the invention provides methods of detecting translocation of one or more virus particles through a polymer. These methods include steps of contacting one or more cells with the one or more virus particles in the presence of the polymer, to produce a combination, maintaining the combination under conditions which mimic the approximate time span for renewal of a mucus layer in vivo and allow translocation through the polymer; and detecting whether the cells are infected with the virus particles, where, if the cells are infected with the virus particles, then the one or more virus particles have translocated through the polymer.

In certain embodiments, the conditions which mimic the approximate time span for renewal of a mucus layer in vivo comprise incubating the combination for about two hours at about 37° C.

In some embodiments, the viral particles comprise one or more plasmids that express a reporter gene in cells infected by the viral particles. In more particular embodiments, the reporter gene is a green fluorescent protein. In other embodiments, the cells are infected with the virus particles is detected by detection of expression of the reporter gene in the one or more cells.

In some embodiments, these methods may further comprise a step of rinsing the cells to remove viruses not attached to the cells.

In other embodiments, these methods may further comprise a step of culturing the one or more cells to allow for the virus particles to infect the one or more cells. In more particular embodiments, the one or more cells are cultured for about 48 hours.

In certain embodiments, the cells are arranged in a monolayer.

In some embodiments, the cells are epithelial cells.

In another aspect, the invention provides a composition comprising about 0.2-2.0% (W/V) non-recombinant mucin, about 200-500 mM NaCl or an equivalent ionic strength salt, and having a pH of about 2-4, wherein a virus exhibits a mean square displacement per second of less than 0.5 μm² on a surface contacted with the composition. In some particular embodiments, the mucin is primarily porcine MUC-5AC. In certain embodiments, the composition is suitable for internal or external administration to a human. In more particular embodiments, the composition is suitable for external, e.g., topical administration to a human.

In another aspect, the invention provides a composition for performing any of the methods disclosed herein. Any composition provided by the invention may in some embodiments be suitable for inhibiting infection by a virus of a cell contacted with the composition or for inhibiting the diffusion or mobility of a virus on a surface contacted with the composition. In more particular embodiments, these compositions may in fact be adapted for and/or used for these purposes, e.g., inhibiting infection by a virus of a cell contacted with the composition and/or inhibiting the diffusion or mobility of a virus on a surface contacted with the composition.

### BRIEF DESCRIPTION OF THE DRAWINGS

FIGS. 1A-1C: Schematic representation of the in vitro infection assay used in this study. (1A) A monolayer of suitable target cells was lined with a biopolymer solution which was then exposed to a small drop of a virus solution. (1B) The cells were incubated with the biopolymer solution and the viruses for 2 h. During this time the viruses could spread through the biopolymer solution and infect the underlying cells. (1C) The biopolymers and remaining viruses were removed by washing with PBS and the cells were incubated for 48 h. All viruses used in this study carried a gene encoding GFP, allowing for distinguishing infected cells (GFP-positive) from uninfected cells (GFP-negative) by flow cytometry.

FIGS. 2A-2B: Basal lamina (ECM) hydrogels trap HPV-16 viruses and act as an infection barrier. (2A) Trajectories of ˜20 s duration for HPV-16 VLPs in the absence of biopolymers and in 0.25% (w/v) ECM. (2B) Percentage of HPV-16-infected HeLa cells using ECM hydrogels at increasing biopolymer concentrations as an infection shield. Error bars denote the error of the mean from three independent experiments.

FIGS. 3A-3B: The diffusive motion of HPV-16 VLPs depends on the microenvironment. (3A) Trajectories of 20 s duration for single HPV-16 VLPs in different biopolymer solutions. (3B) Averaged square displacement of HPV-16 VLPs calculated for a diffusion time of 1 s (see Methods). HPV-16 diffusion was significantly reduced in 1% mucins at pH 7 and massively suppressed in 1% mucins at pH 3. The error bars denote the error of the mean. The number of viral particles analyzed was N=13 for HEPES buffer, N=22 for BSA, N=26 for dextran, N=54 for mucin pH 7 and N=51 for mucin pH 3.

FIGS. 4A-4B: The efficiency of mucin solutions as a shielding layer towards viral infection depends on the mucin concentration. Suitable cells were employed as an infection target, and the percentage of infected (=GFP-positive) cells is determined by flow cytometry. With increasing mucin concentration, the percentage of HeLa cells infected by HPV-16 (4A) and the percentage of MDCK cells infected by influenza (4B) decreased from ˜70-80% to ˜6%. A similar trend was obtained if PBS was used as a mucin hydration buffer instead of HEPES (B). Error bars denote the error of the mean from three independent experiments.

FIGS. 5A-5D: Mucin solutions form more efficient infection shields than other biopolymer solutions. (5A) Pictograms of epithelial cells that are lined with a BSA, dextran or mucin solution. The percentages of infected cells in the presence of these different biopolymers are depicted for the HPV16/HeLa pair in (5B), for the influenza/MDCK pair in (5C), and for the MCV/A549 pair in (5D). As a reference, buffer without biopolymers was used. Error bars denote the error of the mean as obtained from three independent experiments.

FIGS. 6A-6B: The efficiency of mucin solutions to block influenza infection depends on the NaCl concentration of the mucin reconstitution buffer. The percentage of MDCK cells infected by influenza decreased with increasing NaCl concentrations both for 1% (w/v) mucin solutions (6A) and for 0.25% (w/v) (6B) mucin solutions. In the latter case, the enhancement in the protective ability of the mucin solution was much more pronounced. For both data sets, the baseline infection rate of MDCK cells remained unaffected by the amount of NaCl added.

### DETAILED DESCRIPTION OF THE INVENTION

A description of example embodiments of the invention follows.

Mucus is a porous biopolymer matrix that coats all wet epithelia in the human body and serves as the first line of defense against many pathogenic bacteria and viruses. However, under certain conditions viruses are able to penetrate this infection barrier, which compromises the protective function of native mucus. As described herein, it was found that isolated gastric mucin polymers (e.g., isolated porcine gastric mucin polymers), key structural components of native mucus, can protect an underlying cell layer from infection by viruses (e.g., small viruses) such as human papillomavirus (HPV), Merkel cell polyomavirus (MCV), or a strain of influenza A virus. Single particle analysis of virus mobility inside the mucin barrier revealed that this shielding effect is, in part, based on a retardation of virus diffusion inside the biopolymer matrix. The findings provided herein show that purified mucins can be used as a broad-range antiviral supplement to personal hygiene products (e.g., mouth wash, toothpaste, soaps), foods (e.g., baby formula, gum), wound care products (e.g., ointments, bandages) or lubricants to support our immune system.

Specifically, described herein, is the exploration of the antiviral activity of mucins using porcine gastric mucins as an example. It was demonstrated that a solution containing purified porcine gastric mucins efficiently prevented infection of epithelial cells by a broad range of small mucosotropic viruses such as human papilloma virus type 16 (HPV-16), Merkel cell polyoma-virus (MCV) and a strain of influenza A virus. HPV-16 and MCV have comparable diameters of around 50 nm, whereas a typical diameter for an influenza virion is around 100 nm. These sizes are all significantly smaller than the mesh size of native mucus gels. It was found that reconstituted mucins did not compromise the viability of human cervical cell lines or human lung cell lines. Moreover, the results suggest that the ability of mucins to block viral infections is mainly due to a retardation of virus diffusion within the mucin solution.

Accordingly, in one aspect, the invention is directed to a method of inhibiting infection of one or more cells by a virus (e.g., a mucosotropic vius) comprising contacting the one or more cells with a composition comprising biocompatible, purified, non-human gastric mucin. As used herein, “isolated”, “purified” “substantially pure or purified” or “substantially isolated” refers to a mucin that is separated from or enriched relative to the complex cellular milieu in which it naturally occurs, or chemical precursors or other chemicals when chemically synthesized.

“Mucin” and the like is a highly glycosylated protein capable of forming gels, generally comprising an amino and/or carboxy regions that are cysteine-rich and a central region enriched for serine and/or threonine residues and associated O-linked and/or N-linked oligosaccharides. Exemplary mucins include, for example, certain human mucins such as MUC1 (human GeneID No. 4582), MUC2 (human GeneID No. 4583), MUC5AC (human GeneID No. 4586), and MUC5B (human GeneID No. 727897). In certain embodiments, the mucin is a MUC5AC mucin (see, e.g. UniGene IDs 3881294, 1370646, 1774723, 1133368 and HomoloGene 130646), a MUC5B (see, e.g., HomoloGene 124413), a MUC6 (see, e.g., HomoloGene 18768), MUC2 (see, e.g., HomoloGene 130504, 131905, 132025, or 133451) or combinations thereof. In some particular embodiments, the mucin is a secreted mucin, such as MUC5AC, MUC5B, MUC6, and MUC2. In more particular embodiments, the mucin is a gastric mucin, such as MUC5AC, such as a porcine MUC5AC (see, e.g., UnigeneIDs 441382, 5878683; GeneID No. 100170143, and reference sequences AAC48526, AAD19833, and AAD19832). Other mucins suitable for use concordant with the invention include bovine submaxillary mucin (BSM, also known as MUC19; see e.g. GeneID No. 100140959; see HomoloGeneID 130967; see reference protein sequence XP_{—}003586112.1). A mucin-containing composition provided by the invention can be a mixture of one or more mucins (e.g., at least 2, 3, 4, 5, or more different mucins) and, optionally, may be made up of equal or unequal proportions of the different mucins—e.g., a particular mucin may, in certain embodiments, make up at least about 5, 10, 20, 30, 40, 50, 60, 65, 70, 75, 80, 85, 90, 95, 96, 97, 98, 99, or 100% of the mucins in the composition. Preferably, isolated or purified mucin comprises at least about 50%, 75%, 80%, 90%, 95%, 98% or 99% (on a molar basis) of all macromolecular species present.

Any of the individual mucin sequences described in the above annotations can be adapted for use in the invention, as well as variants thereof, e.g., sequences at least 50, 55, 60, 65, 70, 75, 80, 85, 90, 91, 92, 93, 94, 9, 96, 97, 98, 99, or 100% identical to a functional fragment thereof (e.g., comprising about 40, 50, 60, 70, 75, 80, 85, 90, 95, or 100% of the full length of the mature proteins) that is capable of forming a stable mucin surface. Functional variants will generally preserve the function of the conserved domains present in mucins, including VWC (c102515), GHB-like (c100070), TIL (pfam01826) Mucin2_WxxW (pfam13330), VWD (c102516), c8 (c107383), and FA58C (c112042) domains.

Mucins for use in the invention can be chemically or recombinantly (e.g. in CHO or COS cells) synthesized or isolated from a natural source, e.g., from non-human animals. The mucin can be obtained and purified using the methods described herein from any non-human mammal such as a non-human primate, a bovine, a porcine, a canine, a feline, an equine and the like. In a particular aspect, the non-human gastric mucine is porcine gastric mucin. Porcine gastric mucin can be isolated by the methods described in Celli, J., et al., Biomacromolecules 2005, 6 (3), 1329-1333, incorporated by reference in its entirety, preferably omitting the cesium density gradient centrifugation. Advantageously, this method of preparation is efficient and inexpensive.

In the methods of the invention, the concentration of the mucin is typically lower than the mucin concentration of wild type (native) mucin prior to purification—i.e. the in in vivo concentration. In one aspect, the concentration of the gastric mucin is about 4- to about 5-fold lower than the mucin concentration of wild type gastric mucin.

In particular aspect, the mucin concentration is about 0.125, 0.20% (w/v), 0.25% (w/v), 0.30% (w/v), 0.35% (w/v), 0.40% (w/v), 0.45% (w/v), 0.50% (w/v), 0.55% (w/v), 0.6% (w/v), 0.65% (w/v), 0.7% (w/v), 0.75% (w/v), 0.8% (w/v), 0.85% (w/v), 0.9% (w/v), 0.95% (w/v), 1% (w/v), 1.5% (w/v), 2.0% (w/v), 2.5% (w/v), 3.0% (w/v), 3.5% (w/v), 4.0% (w/v), or 4.5% (w/v), or more, when hydrated. In more particular embodiments, the mucin concentration is between about 0.125 to about 2.0% (w/v), e.g. between about 0.2 to about 1.2% (w/v), or more particularly about 0.25 to about 1.0% (w/v)

In yet other aspect, the mucin-containing composition has an acidic or neutral pH (e.g., a pH of about 2, 3, 4, 5, 6 or 7) when hydrated. In more particular embodiments the composition has a pH of about 2 to about 4, e.g. about 2.5 to about 3.5. Ph can be adjusted according to the particular mucin employed.

In another aspect, the mucin-containing composition has a salt concentration of about 20 nM, 40 mM, 60 mM, 80 mM, 100 mM, 120 mM, 140 mM, 160 mM, 180 mM, 200 mM, 220 mM, 240 mM, 260 mM, 280 mM, 300 mM, 320 mM, 340 mM, 360 mM, 380 mM, 400 mM, 420 mM, 440 mM, 460 mM, 480 mM, 500 mM, 520 mM, 540 mM, 560 mM, 580 mM or 600 mM for a 1:1 electrolyte salt when hydrated, or an approximate (e.g., within about 2, 4, 5, 6, 8, 10, 12, 14, 15, 16, 18, 20, 25, or 30%), equivalent ionic strength. In more particular embodiments the salt concentration is about 20 to about 500 mM, e.g., about 150 to about 300 mM; or in other embodiments about 200 to about 500 mM. Suitable salt concentrations can be determined for a particular application by interpolating the desired mucin concentration (by w/v) from the illustrative ranges shown in the examples for 0.25% and 1.0% (w/v) by either linear, quadratic, or higher-order interpolation. In certain embodiments, the reference 1:1 electrolyte salt is sodium chloride—i.e. an approximately equivalent ionic strength can be calculated from the above reference values for NaCl.

As described herein, mucins can be used to inhibit infection of one or more cells by a virus or limit the diffusion (i.e. Brownian motion) or mobility of the virus. In a particular aspect, the virus is a mucosotrophic vius. In certain particular embodiments the virus is not a Norovirus (a group IV virus of the Caliciviridae family) or related virus. In other aspects, the virus has a diameter of about 30 nm, 40 nm, 50 nm, 60 nm, 70 nm, 80 nm, 90 nm, 100 nm, 120 nm, 140 nm, 160 nm, 180 nm, or 200 nm. Examples of viruses include Group I viruses, such as family Papillomaviridae, including human papilloma virus, such as a human papilloma virus type 16 (HPV16) or a Polyomaviridae, such as a Merkel cell polyoma-virus (MCV) or a Herpesviridae, such as a Herpes simplex virus (HSV); Group IV viruses, such as family Flaviviridae, including Hepatitis C virus; Group V viruses, such as a family Orthomyxoviridae virus, including an influenza virus, such as an influenza A virus; a Group VI virus, such as a Lentivirus, including human immunodeficiency virus (HIV); a Group VII virus, such as a Hepadnaviridae, including a Hepatitis B virus. In more particular embodiments, the virus is a Papillomaviridae, Polyomaviridae, Orthomyxoviridae, or Lentivirus. In still more particular embodiments, the virus is a human papilloma virus (e.g., HPV type 16), a Merkel cell polyoma-virus, an HIV virus, or an influenza A virus. In yet more particular embodiments, the virus is an HPV-16, a Merkel cell polyoma-virus, or an influenza A virus

A surface (such as a biological surface) treated in accord with the methods provided by the invention will limit the diffusion or overall mobility of a virus contacted with the treated surface, such that a virus exhibits a mean square displacement (MSD) per second of less than 2.0 μm² on the surface, e.g., 1.9, 1.8, 1.7, 1.6, 1.5, 1.4, 1.3, 1.2, 1.1, 1.0, 0.9, 0.8, 0.7, 0.6, 0.5, 0.4, 0.3, 0.25, 0.2, 0.1, 0.08, 0.06, 0.05, 0.04, Ξm² per second.

In particular aspect, the invention is directed to a composition comprising a biocompatible, purified non-human gastric mucin wherein the concentration of the gastric mucin is about 4- to about 5-fold lower than the mucin concentration of wild type gastric mucin (e.g., about 0.25% (w/v) to about 1% (w/v)), the pH is an acidic or neutral pH (pH 2, 3, 4, 5, 6, or 7), and/or the salt concentration is from about 20 nM to about 500 nM. The invention is also directed to pharmaceutical compositions comprising such compositions. Pharmaceutical compositions, in certain embodiments are of a grade suitable for regulatory approval (e.g. for approval by the U.S. Food ad Drug Administration (FDA)), including USP grade components.

In another aspect, the invention is also directed to a method of detecting translocation of one or more virus particles through a polymer comprising contacting one or more cells (e.g., epithelial cells) with the one or more virus particles in the presence of the polymer, thereby producing a combination. In a particular aspect, the cells are arranged in a monolayer. The combination is maintained under conditions which the approximate time span for renewal of a mucus layer in vivo is mimicked and translocation of the virus through the polymer is allowed. Whether the cells are infected with the virus particles is then detected, wherein if the cells are infected with the virus particles, then the one or more virus particles have translocated through the polymer. Examples of conditions which mimic the approximate time span for renewal of a mucus layer in vivo include incubating the combination for about two hours at about 37° C.

In a particular aspect, the viral particles can comprise one or more plasmids that express a reporter protein (green fluorescent protein) in cells infected by the viral particles. In this aspect, whether the cells are infected with the virus particles is detected by detection of expression of the reporter gene in the one or more cells.

The method can further comprise rinsing the cells to remove viruses not attached to the cells. In addition, the method can also comprise culturing the one or more cells to allow for the virus particles to infect the one or more cells (e.g., culturing the one or more cells for about 48 hours).

As will be appreciated by those of skill in the art, the use of mucins to inhibit viruses can be applied in a variety of ways. For example, the mucins can be used as a broad-range antiviral supplement to personal hygiene products (e.g., mouth wash, toothpaste, soaps), foods (e.g., baby formula, gum), wound care products (e.g., ointments, bandages) or lubricants to support our immune system. Additional applications include applying the mucin compositions provided by the invention to human work surfaces, such as doorknobs, table tops, faucet handles, toilets, phones, et cetera. The protective effects of the mucin compositions provided by the invention can also be useful in filtration applications, such as, for example, ductwork, and filters for ductwork, e.g., in environment control systems such as heating and air conditioning, e.g. enclosed spaces, such as in automobiles, trains, airplanes, subways, et cetera. A subject to be treated by the methods provided by the invention, or to be administered the compositions provided by the invention, can be any subject. A “subject” refers to a mammal, including primates (e.g., humans or monkeys), cows, sheep, goats, horses, dogs, cats, rabbits, guinea pigs, rats, mice or other bovine, ovine, equine, canine, feline, rodent or murine species. Examples of suitable subjects include, but are not limited to, human patients. In particular embodiments, the subject to be treated by the methods provided by the invention is human and can be male or female and may be at any stage of development: e.g., prenatal, neonatal, infant, toddler, grade-school-age, teenage, early adult, middle-age, or geriatric, e.g., at least or about 1, 2, 3, 4, 5, 6, 7, 8, 9, 10, 15, 18, 20, 21, 25, 30, 35, 40, 45, 50, 55, 60, 65, 70, 75, 80, 85, 90, 95, 100, 105 years old, or more. Particular biological surfaces to be treated by the methods of the invention include joints, such as articular joints, as well as mucosa, including mucosa of the eyes, mouth, nose, the urogenital genital tract (such as the female genital tract), the respiratory tract (e.g., the lungs by, for example, inhalation), or digestive tract, as well as combinations of these. Other biological surfaces include the skin. Biological surfaces may either be intact (e.g. healthy) or injured (e.g. by burn, rash, irritation, cut, tear, disease, et cetera.

Any of the compositions described as suitable for the methods provided by the invention are encompassed as an additional aspect of the invention. The compositions can be adapted for the particular application, such as pharmacological or nutrient-supplemental purposes.

Any suitable delivery mode known to the skilled artisan is appropriate for delivering the compositions provided by the invention. Exemplary delivery modes include topical solutions, gels, lotions, creams, ointments, or pastes; other modalities includes oral administration, e.g., for stomach or intestinal delivery; enemas; or injection. For example, the compositions provided by the invention can, in some embodiments, be incorporated in hygiene products, lotions, aqueous solutions (e.g., eye drops), gels; to affect saliva, in some embodiments, they can be provided in toothpaste; for embodiments to treat stomach mucosa, edible forms, such as yogurt, can be used.

Thus, the invention is also directed to a composition, such as a pharmaceutical composition, comprising one or more of the compositions provided by the invention. For instance, the compositions can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, et cetera.

Methods of introduction of these compositions include, but are not limited to, intradermal, intramuscular, intraperitoneal, intraocular, intravenous, subcutaneous, topical, oral and intranasal. The pharmaceutical compositions of this invention can also be administered as part of a combinatorial therapy with other compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, that notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the compounds can be separated, mixed together in any combination, present in a single vial or tablet. Compounds assembled in a blister pack or other dispensing means is preferred. For the purpose of this invention, unit dosage is intended to mean a dosage that is dependent on the individual pharmacodynamics of each compound and administered in FDA approved dosages in standard time courses.

### EXEMPLIFICATION

### Materials and Methods

### Proteins and Hydrogel Reconstitution

Bovine serum albumin (BSA), matrigel (basal lamina, a subtype of the extracellular matrix=ECM) and dextran (MW: 2 MDa) were obtained from Sigma-Aldrich (St. Louis, Mo.), commercial mucins were purchased from Sigma Aldrich and from NBS Biologicals (Huntingdon, UK). The complex ECM Matrigel has been purified from the basal membrane of the Engelbreth-Holm-Swarm sarcoma of mice and is widely used as a model system for native ECM. A 1% (w/v) stock solution of ECM was thawed on ice and diluted with Dulbeccos's Modified Eagle Medium (DMEM, Invitrogen, Carlsbad, Calif.) where necessary. Once applied to cells, it was incubated for 30 min at 37° C. to induce gelation. Porcine gastric mucins were purified from scrapings of fresh pig stomachs essentially as described in Celli, J., et al., Biomacrmolecules 2005, 6(3), 1329-1333, with the exception that the cesium chloride density gradient ultracentrifugation was omitted.

To determine the molecular composition of the mucin preparation in more detail, its content was analyzed at the “Harvard Microchemistry & Proteomics Analysis Facility” by microcapillary reverse-phase HPLC nano-electrospray tandem mass spectrometry on a Thermo LTQ-Orbitrap mass spectrometer. This instrument is capable of acquiring individual sequencing spectra at high sensitivity (<<1 femptomol) for multiple peptides in the chromatographic run. The spectra were then correlated with known sequences using the algorithm Sequest (Yates, J R, et al., Anal Chem 1995, 67(8), 1426-1436). The analysis showed that the mucin preparation contained the following mucins: MUC5AC, MUC2, MUC5B and MUC6. As expected for the stomach mucosa, MUC5AC seemed to be the major component of the purification. Other proteins within the mucin preparation included histones, actin, and albumin. Yet, as this mass spec technique was not suitable to determine exact protein concentrations, the relative concentrations of all those proteins could not be established. For reconstitution, lyophilized mucins were hydrated overnight at 4° C. in distilled water. The homogeneous mucin solutions were buffered to the desired pH with acetate buffer calibrated to pH 3 or HEPES buffer calibrated to pH 7. The final buffer concentration in the mucin solution was 20 mM and the ionic strength was adjusted to 20 mM with NaCl. BSA, dextran and commercial mucins were treated the same way as the purified mucins and hydrated in either pH 7 HEPES buffer containing 20 mM NaCl, or in PBS.

### Virus Particles

HPV-16 and MCV reporter vectors (pseudoviruses) carrying GFP reporter plasmids and fluorescently labeled virus-like particles (VLPs) were generated according to previously-reported methods (Neumann, G., et al., Nature 2009, 459(7249), 931-939). In brief, 293T cells were co-transfected with expression constructs encoding the capsid genes with or without a reporter plasmid encoding GFP. Plasmids used in this work are available through Addgene.org and detailed protocols are posted at <home.ccr.cancer.gov/LCO/>. The influenza virus used here was the A/WSN/1933 (H1N1) strain, which is a commonly used influenza lab strain. For this study, the virus had been modified such that the PB1 segment carries the green fluorescent protein (GFP) in place of the PB1 coding sequence, as described in Loyo, M., et al., Int J Cancer (2010), 126(12), 2991-2996. As a consequence, these influenza viruses were only able to replicate in engineered cell lines that expressed the PB1 protein.

HPV-16 VLPs were conjugated to Alexa Fluor 488 according to Alexa Fluor 488 Protein Labeling Kit (Invitrogen #A10235) instructions. Clarified cell lysate containing VLPs was diluted with water to a total protein concentration of 3 mg/ml, adjusted to 0.1 M sodium bicarbonate and added to a vial of Alexa Fluor 488 reactive dye provided with the kit. The conjugated VLPs were adjusted to neutral pH with sodium phosphate and then purified over an Optiprep gradient. Infectivity monitoring using an encapsidated Gaussia Luciferase plasmid indicated no detectable alteration of the particle-to-infectivity-ratio of Alexa Fluor 488 conjugated VLPs, indicating that the dye conjugation reaction did not have a major impact on capsid protein function.

### Cell Culture

Human cervical cells (HeLa) were cultured in DMEM (Invitrogen, Carlsbad, Calif.) supplemented with L-glutamine, 10% fetal bovine serum (FBS), 4.5 g/L glucose, and 25 U/mL penicillin, 25 μg/mL streptomycin at 37° C. in a humidified atmosphere at 5% CO2. The human alveolar epithelial cell line A549 was cultured in RPMI medium (Invitrogen, Carlsbad, Calif.) supplemented with L-glutamine, 5% FBS, 4.5 g/L glucose, and 25 U/mL penicillin, 25 μg/mL streptomycin at 37° C. in a humidified atmosphere at 5% CO2. As an infection target for the influenza virus, a variant of Madin-Darby canine kidney cells, MDCK-SIAT1-CMV-PB1, was used. These highly adherent epithelial cells have been modified by lentiviral transduction to constitutively express the PB1 protein under a CMV promoter to allow the modified influenza viruses used in this study to efficiently replicate inside these cells. MDCK cells were cultured in DMEM (Invitrogen, Carlsbad, Calif.) supplemented with L-glutamine, 10% fetal bovine serum (FBS), 4.5 g/L glucose, and 25 U/mL penicillin, 25 μg/mL streptomycin at 37° C. in a humidified atmosphere at 5% CO2. Cell counts were performed using a hemocytometer.

### In Vitro Infection Assay

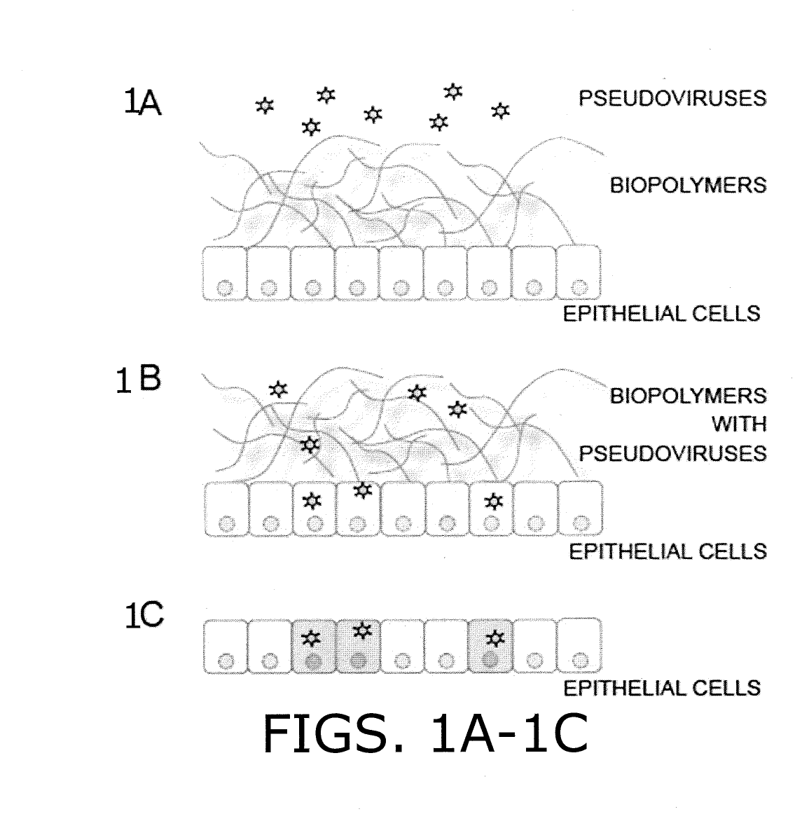

For in vitro infection, 5×10⁴ HeLa cells, 7.5×10³ A549 cells, or 3×10⁴ MDCK-SIAT1-CMV-PB1 cells were seeded into each well of a 96-well microtiter plate (BD Biosciences, San Jose, Calif.) and allowed to adhere overnight. For MDCK SIAT1-CMV-PB1 cells, three hours prior to infection the DMEM was replaced by OptiMem media (Invitrogen, Carlsbad, Calif.) supplemented with 0.01% FBS, 0.3% BSA, 100 U/mL Penicillin, 100 μg/mL Streptomycin, and 100 μg/mL CaCl2. For infection, the cell culture medium was removed, and replaced with 50 μL of a biopolymer solution (ECM, mucin, BSA, or dextran). As controls, cells were lined with DMEM, PBS or HEPES buffer (20 mM HEPES, 20 mM NaCl, pH 7). For infection, 5 μL of virus solution was carefully added to each well (FIGS. 1A-1C) and incubated for two hours at 37° C. This mimicked the approximate time span after which a mucus layer is renewed in vivo, owing to the synthesis of fresh mucins on the epithelial surface and subsequent shedding of the “old” mucus layer together with trapped particles. In the in vitro setup described herein, the virus particles were allowed to translocate through the biopolymer solution. This process is driven by a combination of diffusion, turbulences created by adding a drop of virus solution onto the biopolymer layer, and thermal convection effects. After this incubation step, the cells were rinsed three times with 150 IL PBS. This washing step removed the biopolymer solution together with any viruses that have not yet attached to the cell monolayer. Then, 150 μL cell culture medium was applied and the cells were cultured for 16 hrs (for MDCK-SIAT1-CMV-PB1) or for two more days (for HeLa and A549), respectively, to allow for expression of GFP. For FACS analysis, the adherent cells were harvested by trypsinization and analyzed using a LSRII flow cytometer (BD Biosciences, San Jose, Calif.) or an Accuri C6 flow cytometer (Accuri Cytometers, Cambs, UK). Uninfected control cells were used to define the base-line autofluorescence of each cell batch. Thresholds in the analysis software (FACSDiva version 6.1.2, BD Biosciences, San Jose, Calif., or CFLow Plus, Accuri Cytometers, Cambs, UK) were chosen in such a way that 0.1% of the control cells were counted as GFP-positive. Relatively high viral doses were used to minimize assay variability we sometimes observed with multiplicities of infection below 1. All experiments were performed in triplicates.

### Cell Toxicity Assay

For assessing putative cytotoxic effects of mucins, HeLa cells were incubated with a 1% (w/v) pH 7 mucin solution for 2 hrs, followed by washing with PBS. The cells were then incubated with DMEM for 48 hrs. Then, the percentage of viable cells was determined using a live/dead kit for mammalian cells (Invitrogen, Carlsbad, Calif.). In brief, cells were stained with two dyes, calcein and Ethidium homodimer-1, which emit green and red fluorescence, respectively. Cells that emit green fluorescence only are considered viable, whereas red fluorescence is a marker for cytotoxic effects. 2 μM calcein and 2 μM Ethidium homodimer-1 were suspended in the appropriate cell media and incubated with the cells for 20 min. Images were acquired on an Axio Observer microscope (Zeiss, Oberkochen, Germany) with a EC-Plan Neofluar 10×0.3 NA lens (Zeiss). Cell counts were performed with the image analysis software ImageJ using a cell count plug-in.

### Single Particle Tracking

For single particle tracking experiments, mucins (or dextran, or BSA) were hydrated as described before. Fluorescently labeled HPV-16 particles were added to each biopolymer solution, and ˜30 μL of the virus/biopolymer solution was placed into an open 10 mm×10 mm square of vacuum grease which has been deposited onto a glass slide. This custom made sample chamber was then sealed with a cover slip. Brownian motion of virus particles was followed at room temperature on an Axio Observer microscope (Zeiss, Oberkochen, Germany) with an EC Plan 40×0.75 NA PH2 objective (Zeiss) using fluorescence microscopy. Movies were acquired with a digital camera (ORCAR2, C10600; Hamamatsu, Hamamatsu City, Japan) at 10 frames/s and processed with the software OpenBox. For diffusing virus particles it is highly difficult to obtain coherent trajectories r(t)=(x(t), y(t)) due to the weak contrast within the biopolymer solutions and the high virus mobility in certain samples. Thus, the calculation of a full MSD curve, MSD(τ)=Σ[r(iΔt+τ)−r(iΔt)]², as typically applied to single particle tracking data is not feasible here. Instead, from the movies the squared distance δ²=[r(t₀+1s)−r(t₀)]² a given virus particle travels by diffusion within 1 s of observation time—corresponding to 10 frames, i.e. a time span that most of the virus particles can be followed coherently, was measured. The average <δ²> was then calculated by pooling measurements from each sample, i.e. data for up to 50 different virus particles and different starting time points t₀. This yielded a quantity that represents the mean square displacement of the particle ensemble for a lag time of τ=1 s, <δ²>=MSD(1s). It should be noted that, due to the limited statistics, this quantity could not be used to determine an accurate diffusion coefficient. It gave, however, a reasonable measure for comparing the diffusion behavior of the VLPs in different micro environments.

### Viscosity Measurements

The dynamic viscosity of the biopolymer solutions was measured with a stress controlled rheometer (AR-G2, TA instruments, New Castle, USA) with a 40 mm cone-plate geometry (2° cone angle) and 56 μm truncation distance. Approximately 500 μL of a biopolymer solution was loaded onto the rheometer, subjected to a shear rate ramp in the range of 100/s-1000/s and the viscosities measured in this shear rate range were averaged.

### Results

Hydrogels can Protect Cells from Viral Infection by Trapping the Viruses in the Biopolymer Matrix

For initial experiments reporter pseudovirions based on HPV-16, an HPV type that causes a majority of cases of cancer of the uterine cervix, were used. As targets for HPV-16 infection HeLa cells, which originate from human cervical tumor tissue, were chosen. In vivo, prior to infection of the target cells, HPV-16 attaches to the basal lamina, a complex multi-component hydrogel that supports the basal cells of the cervical epithelium. This attachment process requires binding interactions between the HPV-16 capsid proteins and the glycosaminoglycan heparan sulfate, which is one of the main components of the basal lamina.

Indeed, this adsorption of the virus was able to be reproduced in vitro by using a basal lamina hydrogel (here referred to as extracellular matrix (ECM), see Materials). This hydrogel was a complex mixture of the biopolymers collagen IV, laminin and heparan sulfate. In the concentration regime used here, the ECM hydrogel had a mesh size on the order of a few microns. Previous experiments have shown that particles much smaller than this mesh size can be efficiently immobilized in the ECM biopolymer matrix. This immobilization effect is, in part, established by the heparan sulfate glycosaminoglycan and due to electrostatic binding interactions between the diffusing particles and the hydrogel biopolymers.

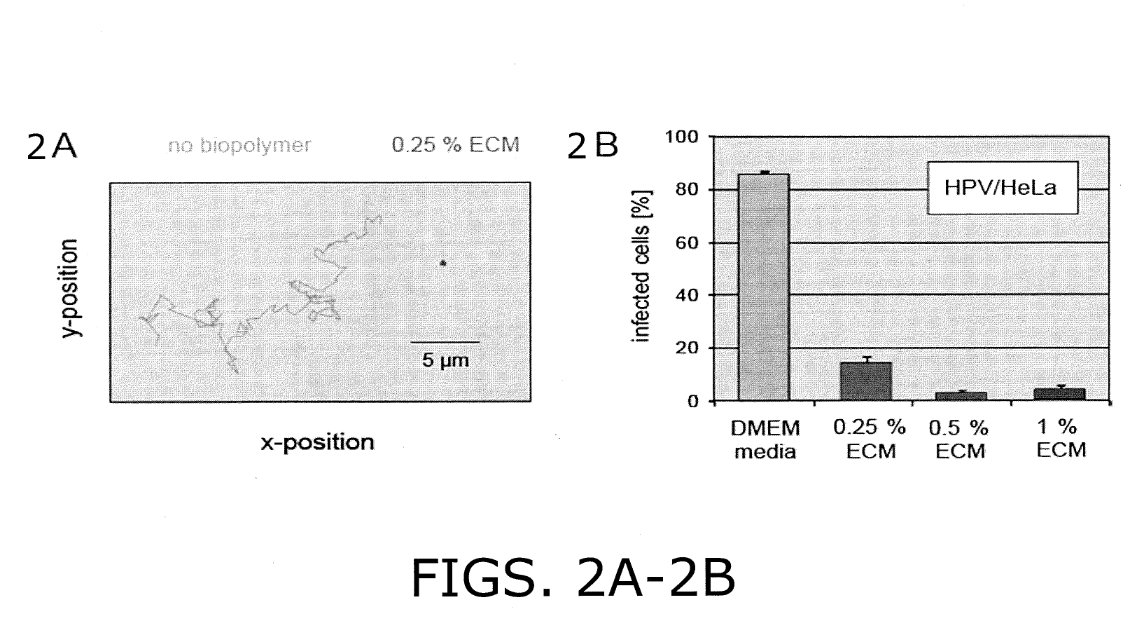

Here, it was found that a 0.25% ECM hydrogel also suppressed the diffusive motion of HPV-16 pseudovirions (FIG. 2A), and a very low diffusion coefficient of D_{HPV,ECM}=(8±2)×10⁻⁴ μm²/s was measured. This value is ˜10,000 times smaller than the corresponding value for pseudovirion diffusion in buffer control conditions which was calculated to be D_{HPV,water}=9.6 μm²/s using the Stokes-Einstein relation, D(R)=k_{B}T/(6πηR). There, k_{B}T denotes the thermal energy, η the viscosity of water and R the radius of the HPV 16 virus particle. It was then hypothesized that a hydrogel, which can trap virus particles inside the biopolymer matrix, should also be able to protect underlying cells from viral infection. To test this hypothesis, a HeLa monolayer was lined with either DMEM or with an ECM hydrogel and then exposed the culture to HPV-16 pseudoviruses (FIGS. 1A-1C and methods). Indeed, a 0.25% (w/v) ECM gel reduced the percentage of infected HeLa cells about 6-fold compared to the positive control (FIG. 2B). The shielding efficiency of ECM became stronger at higher biopolymer concentrations where the HPV-16 infection rate is reduced to only a few percent. Together, these data demonstrate that ECM hydrogels have the capacity to effectively prevent viruses from infecting underlying cells, and that this is achieved by trapping the viruses in the biopolymer matrix.

### Reconstituted Mucin Solutions do not Compromise the Viability of HeLa Cells

Whether purified porcine gastric mucins can also serve as an anti-viral infection barrier was then tested. However, before assessing the impact of porcine gastric mucins on the diffusion behavior and infectivity of HPV-16, it was verified that reconstituted mucin solutions are non-toxic for HeLa cells (see Methods). It was found that HeLa cells exposed to the mucin solution showed a viability that was comparable to cultures exposed to the HEPES buffer control (Table). Similar results were obtained for commercial porcine gastric mucins purchased from Sigma Aldrich. However, commercial mucins obtained from another vendor (NBC Biologicals, Huntingdon, UK) appeared to induce strong cytotoxic effects as the corresponding percentage of viable cells was very low. It was concluded that the manually purified mucin solutions constituted a biocompatible material that does not compromise the viability of HeLa cells.

### Mucin Solutions Retard HPV-16 Diffusion

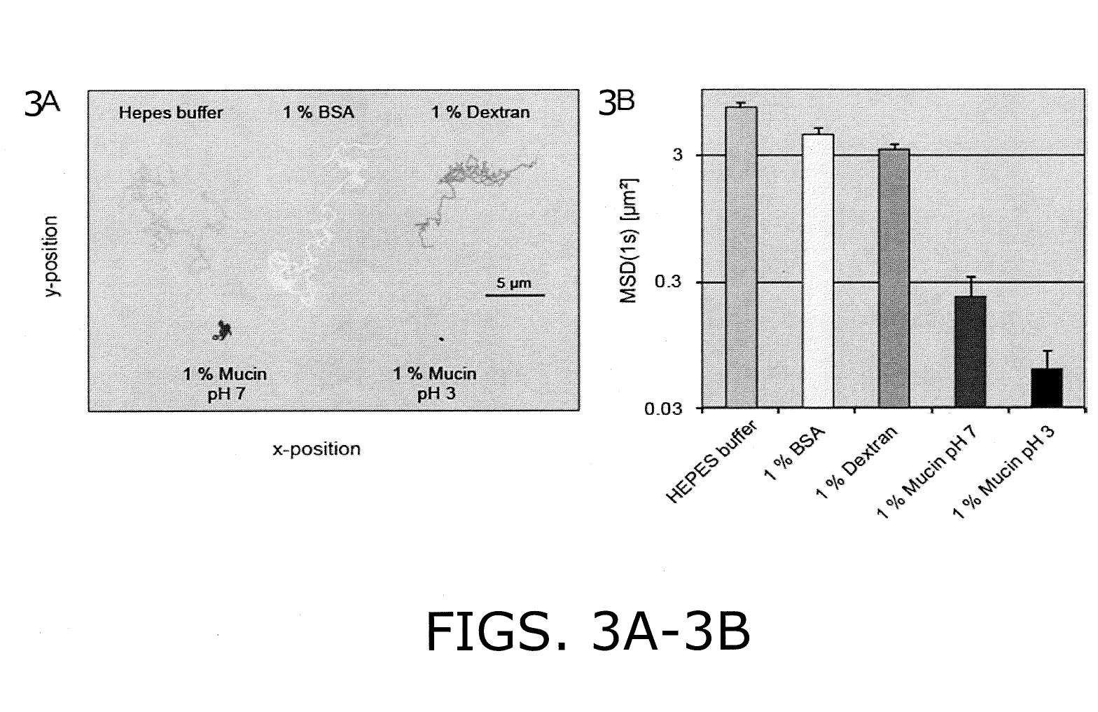

If porcine gastric mucins act as an infection barrier towards HPV-16 viruses, then trapping of virus particles inside the mucin matrix would be a possible mechanism to achieve this goal, similar to what was observed for ECM hydrogels. Thus, next analyzed was the diffusion behavior of individual fluorescently labeled HPV-16 VLPs in mucin solutions using single particle tracking microscopy (FIG. 3A and Methods). In HEPES buffer, HPV-16 diffused rapidly and it was difficult to follow a given VLP for an extended time period as most of the virus particles left the focal plane during the observation time. In contrast, in a 1% mucin solution, the diffusive motion of HPV-16 VLPs was significantly suppressed. As depicted in FIG. 3A, this retardation of HPV-16 diffusion is even more pronounced when the pH level of the mucin solution is lowered from pH 7 to pH 3. At the acidic pH, where the mucin biopolymers form a hydrogel, the VLPs appeared to be completely immobilized. Importantly, this lock-down of virion mobility appeared to be a global effect since mobile VLPs were not detected in the pH 3 sample.

To better quantify the observed differences in the VLP diffusion behavior, the mean square displacement was determined for an ensemble of up to 50 virus particles per condition at a lag time of 1 s (see Methods). As depicted in FIG. 3B, this ensemble quantity reflected the pronounced differences observed in the trajectories of single virus particles. In other words, mucin solutions were able to efficiently trap the VLPs both at neutral and acidic pH. This indicates that mucin solutions should also be able to employ this trapping of HPV-16 virus particles to act as an infection shield in our in vitro infection assay.

### Mucin Solutions Pose a Barrier Towards HPV-16 Infection

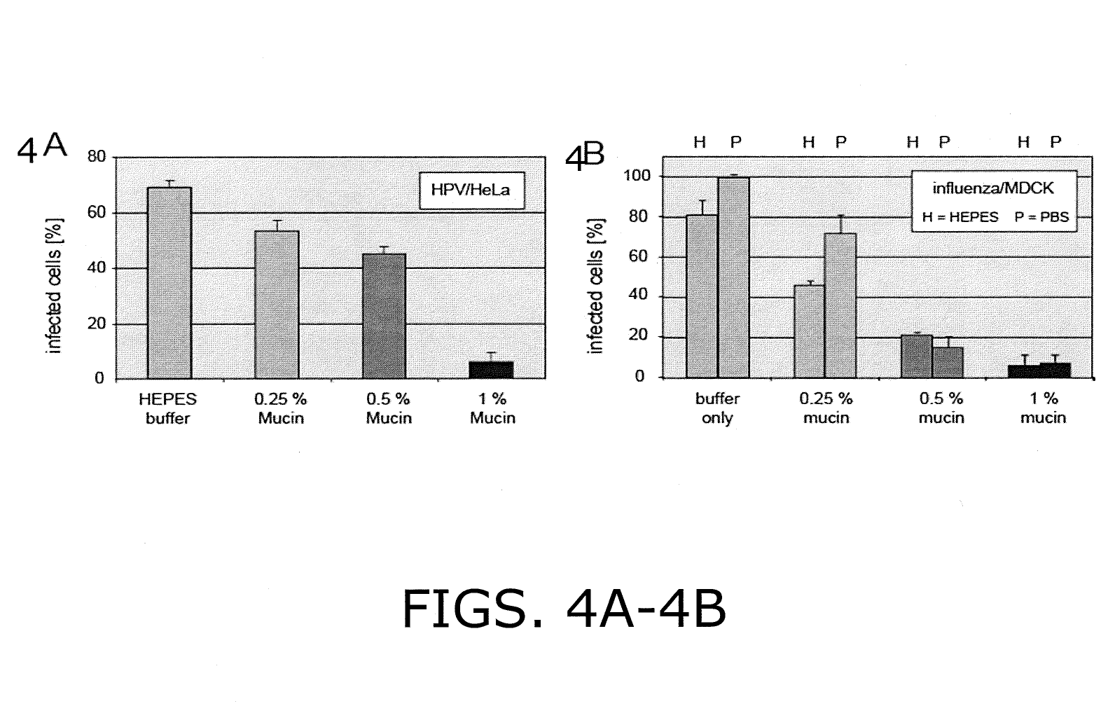

In the next step, the performance of porcine gastric mucins as a barrier towards viral infection was evaluated. When HEPES buffer was used as the sole protective layer, more than 65% of the cells were infected (FIG. 4A). In contrast, when the cells were covered with a 1% (w/v) mucin solution prior to virus inoculation, the proportion of infected cells was decreased to ˜6%. The shielding efficiency of the mucin solution depended on the mucin concentration used (FIG. 4A), confirming previous findings that the permeability of mucin solutions can be regulated by the mucin concentration, in analogy to native mucus. This demonstrated that purified mucins can indeed pose an efficient barrier towards HPV-16 infection. Interestingly, commercial porcine gastric mucins obtained from Sigma Aldrich were less efficient in reducing HPV-16 infection when used at a similar concentration as the manually purified mucins described herein (Table). Similarly, also particle diffusion experiments have shown that commercially purified mucins do not establish the same diffusion control as native intestinal mucus. The findings herein indicate that the detailed mucin purification protocol and mucin biochemistry are important for the performance of mucins as a barrier towards viral infection.

Mucin Solutions Form More Efficient Diffusion and Infection Barriers than Other Biopolymer Solutions

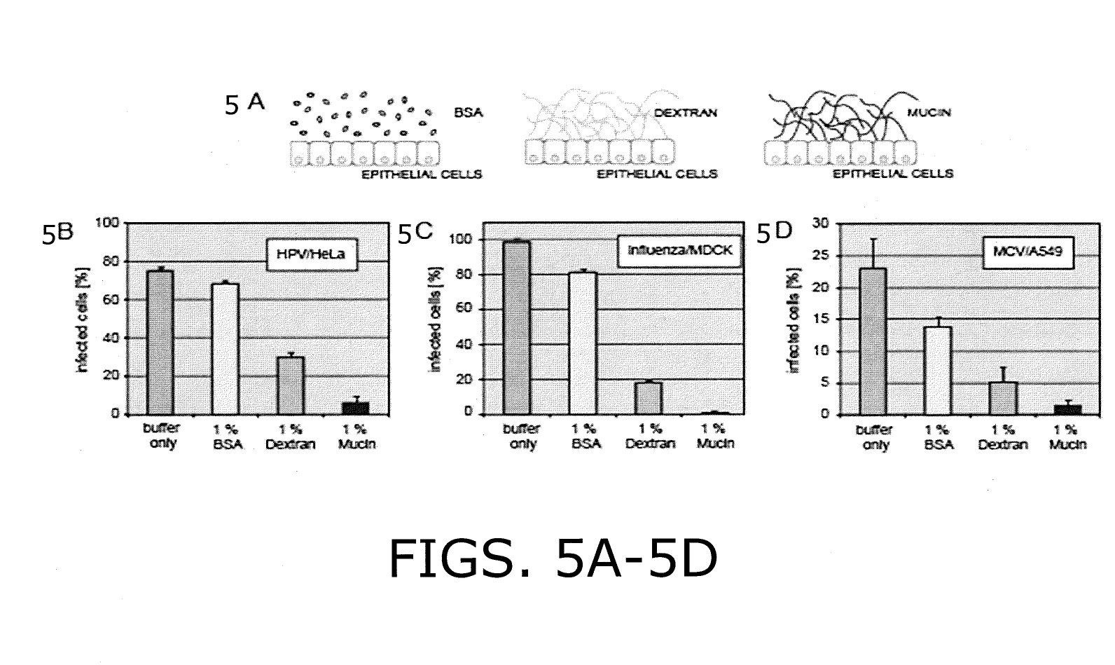

Can any solution of macromolecules protect cells from viral infections or are certain biopolymer properties required to achieve this effect? To tackle this question, the results obtained for 1% (w/v) mucin solutions were compared to two other biopolymer solutions: a bovine serum albumin (BSA, MW ˜66 kDA) solution and a dextran solution (MW ˜2 MDa), which were both prepared at the same concentration as the mucin solution, i.e. at 1% (w/v) (FIG. 5A). When used as a shielding layer in the in vitro infection assay described herein, BSA did not cause a major reduction in the infectivity of HPV-16, and dextran was only moderately inhibitory (FIG. 5B). Moreover, neither the BSA solution nor the dextran solution appeared to significantly inhibit HPV-16 diffusion (FIGS. 3A and 3B). This indicated that the inhibitory effect of dextran observed in FIG. 5B was not simply due to a reduction of virus diffusion, but rather that dextran likely modestly inhibit a subsequent step in the infection process. These results indicate that unique chemical/physical properties of the mucin biopolymer are needed for efficient virus trapping and infection blocking.

### Mucin Solutions Also Pose a Barrier Towards Influenza and MCV Infection

Next investigated was whether the shielding effect of mucin solutions is specific for HPV-16 or whether mucins can prevent other small viruses from infecting epithelial cells as well. To address this question, the in vitro infection experiments described before were repeated, but this time using influenza viruses (100 nm). Influenza A belongs to the virus family Orthomyxoviridae and infects the epithelia in the respiratory tract of humans. The introduction of new influenza subtypes into the human population can cause pandemics, including the deadly 1918 pandemic (which is estimate to have killed over 40 million people worldwide) and more recently the 2009 swine-origin H1N1 pandemic. As targets for infection with the A/WSN/33 (H1N1) strain of influenza used in this study, modified canine kidney cells (MDCK-SIAT1-CMV-PB1, see Methods) were chosen. The viability of this cells type was also unaffected by our 1% (w/v) mucin solution (Table). With the in vitro infection assay described herein, a similar ability of mucins to block influenza infection as observed for HPV-16 pseudoviruses was found (FIG. 4B). A comparable dependency of this infection barrier efficiency on the mucin concentration was obtained when HEPES or PBS was used as a buffer for mucin reconstitution, respectively (FIG. 4B). Furthermore, the inhibitory profiles of BSA, dextran and mucin towards influenza infection resembled those obtained for the HPV/HeLa cell pair, with mucins being much more efficient than BSA or dextran (FIG. 5C).

As a third virus species, the Merkel cell polyomavirus (MCV, ˜50 nm), which is believed to infect both the skin and the oral mucosa was also investigated. MCV is a member of the viral family Polyomaviridae and is thought to trigger Merkel cell carcinoma, an aggressive form of skin cancer. MCV pseudoviruses can successfully transduce A549 cells, a cell line established from a human lung epithelial tumor. With this virus/cell pair, the same trend in the protective ability of BSA, dextran, and mucin solutions as for the HPV16/HeLa combination was observed (FIG. 5D). In agreement with the previous findings on HeLa and MDCK cells, it was also observed that the viability of A549 lung cells was not compromised by exposure to a 1% (w/v) mucin solution (Table) underscoring the bio compatibility of the purified mucin biopolymers. Together, these data demonstrate that the mucin biopolymer matrix imposes an efficient infection barrier not only towards HPV-16 but also towards influenza and MCV.

### High NaCl Concentrations Increase the Barrier Function of Mucins

Rinsing the nose or the oral cavity with saline solutions is a traditional household remedy for the common cold. Thus, in a final step, it was asked how different concentrations of sodium chloride in the mucin hydration buffer would affect the inhibitory activities of mucin solutions towards viral infection. For this set of experiments the influenza/MDCK cell pair was chosen, as only MDCK cells showed strong enough adhesion to allow for performing the in vitro infection assay in the presence of high NaCl concentrations.

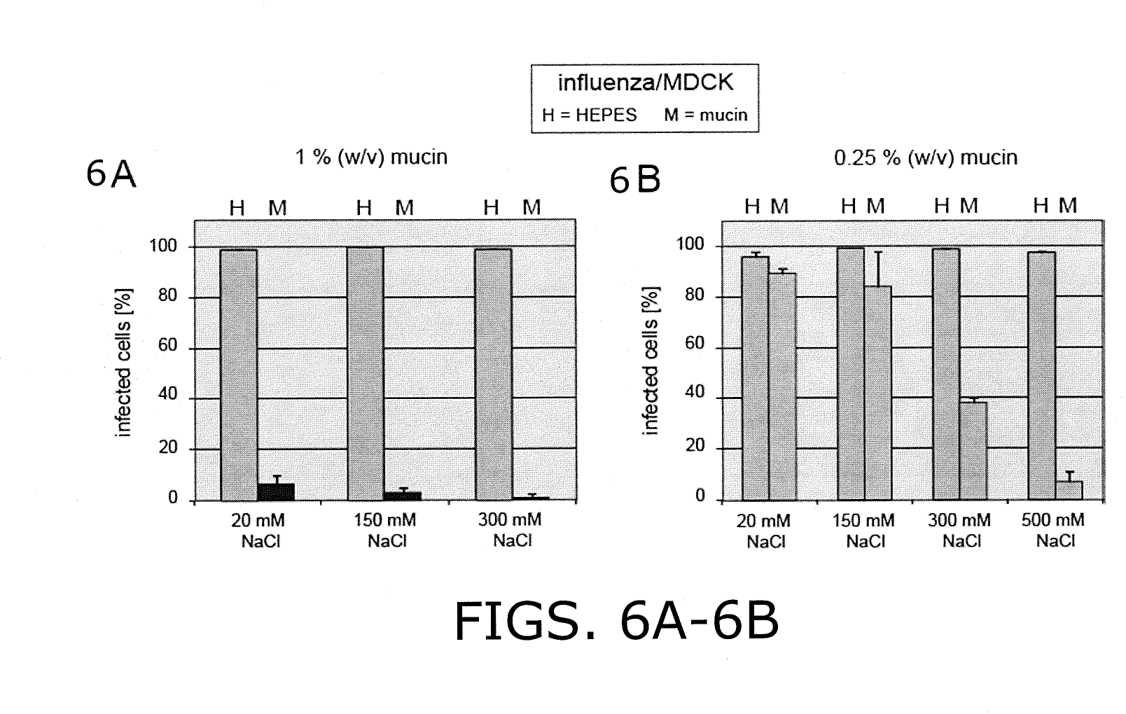

When mucins were hydrated in low salt HEPES buffer (20 mM NaCl), the concentration dependent inhibitory activity of mucins was comparable to the situation when PBS (140 mM NaCl and minor amounts of divalent ions) was used for mucin hydration (FIG. 4B). Accordingly, also HEPES buffer containing 150 mM NaCl returned comparable results when used as a hydration medium for 0.25% (w/v) and 1% (w/v) mucin solutions (FIGS. 6A and B). However, when the NaCl concentration of the HEPES buffer was increased to 300 mM (FIG. 6A), a significant enhancement of the influenza inhibition efficiency provided by a 1% (w/v) mucin solution was detected. This effect is not due to an altered base-line infectivity or reduced virus stability, as the control samples were unaffected by the change in the ionic strength of the mucin hydration buffer. A similar trend, albeit much stronger, was observed when the NaCl concentration was varied in a 0.25% (w/v) mucin solution. At this low mucin concentration, the inhibitory activity of the mucin solution was drastically enhanced by raising the NaCl concentration (FIG. 6B). Surprisingly, at 500 mM NaCl, the low concentration mucin solution was as efficient in reducing influenza infectivity as the 1% (w/v) mucin solutions discussed before. These results demonstrate that the ability of mucin solutions to act as an anti-viral infection shield can be strengthened by high NaCl concentrations.

### Discussion

The data reveal that purified gastric mucins (e.g., purified porcine gastric mucins) can efficiently shield an underlying cell monolayer from infection by viruses as small as 50 nm in diameter. This effect can be achieved with mucin concentrations as low as 0.25-1% (w/v) which is 4-5 fold lower than the mucin concentrations found in native mucus. Data obtained at the single particle level suggest that the barrier function of mucin solutions is achieved in part by suppressing the mobility of virus particles and trapping them inside the biopolymer matrix. Previous studies on virus diffusion in native mucus have suggested that this immobilization effect is based on adhesive interactions with certain mucus components, and a similar mechanism—in addition to geometric hindrance effects—might also be responsible for the virus trapping described here. However, owing to the lower mucin concentration, the mesh size of the reconstituted mucin system described herein should be larger than the literature value of 300 nm reported for native cervical mucus and thus significantly larger than the diameter of the viruses studied here. This indicates that geometric hindrance is not likely to be the main mechanism by which mucin solutions trap virus particles.

Interestingly, the antiviral activity of mucins was still observable when the mucin layer was removed from the cells by washing prior to virus inoculation (data not shown). It is possible that mucin polymers remain on the cellular surface after the washing step, and that these could act as a passivation agent by blocking the cell receptors required for virus binding thus preventing successful cell infection. Such a cell membrane passivation mechanism might also explain the modest inhibitory effect of dextran and even BSA mentioned earlier and could constitute a second anti-viral defensive mechanism provided by mucins besides virus trapping. Alternatively, this could indicate that, indeed, the interaction with mucins affects cell physiology, perhaps by modulating the cytoskeleton, or by other, entirely unrelated cellular mechanisms that render cells more resistant toward viral infection. Shown herein is that the trapping of HPV-16 inside the mucin matrix is more efficient at low pH. This agrees with previous findings on particle diffusion and particle translocation experiments in reconstituted mucin solutions and underlines the possibility to tune the permeability of this biopolymer based diffusion barrier by pH. It is emphasized that our single particle tracking assay might still underestimate the virus trapping effect established by mucins: the residual mobility observed for HPV viruses in mucin solutions could represent thermal undulations of the mucin biopolymers to which the virus particles are bound, rather than a true local diffusion of the virus particles.

Which components of reconstituted mucin solutions could be responsible for trapping virus particles inside the biopolymer matrix? One commonality of the viruses studied here is their interaction with sugar groups during the infection process. HPV-16, for example, targets negatively charged heparan sulfate chains in the ECM or on the cell surface. MCV appears to use heparan sulfate and sialyated glycans as receptors. Influenza attaches to cells via binding of the hemagglutinin protein to sialic acid moieties on the host cell. Mucins contain polyanionic sugars including sialic acid, which may serve as decoys for the various receptors utilized by the three virus families studied herein. It is interesting to note that commensal and pathogenic microbes appear to specifically target such sugar moieties when they are trying to weaken the native mucus barrier by enzymatic degradation, and that the influenza neuraminidase protein can cleave sialic acid moieties. This supports the notion that those sugar moieties indeed play an important role in the defense mechanism of mucins towards pathogens. Whereas the detailed mechanistic and molecular principles that establish the adhesive effects between viruses and mucins are still to be deciphered, the adsorption of the virus particles to mucins could, at least in part, be due to relatively non-specific electrostatic interactions with either the sugar moieties or with the protein backbone of the mucin biopolymers. Electrostatic interactions have already been suggested to contribute to the trapping of polystyrene particles inside reconstituted mucin solutions. However, other physical interactions such as van-der-Waals forces or hydrogen bonds could also cause trapping of viruses inside the mucin matrix. Indeed, the finding described herein that high NaCl concentrations strengthen the barrier function of mucin solutions demonstrates that electrostatic binding interactions are not sufficient to explain the barrier properties of mucins towards virus particles. At high ionic strength, the electrostatic interactions between the virus capsids and the mucin biopolymers should be sufficiently weakened by Debye screening. Thus, if binding of influenza viruses by mucins were based on electrostatic interactions only, then high ion concentrations would be expected to reduce the barrier function of mucins towards influenza rather than strengthen it.

In native mucus such as saliva, certain ions such as Ca²⁺ increase the macroscopic viscosity of the hydrogel and retard the diffusion of tracer particles. The viscosity of the mucin solutions described herein, however, is reduced by (12±7) % at 500 mM NaCl compared to low salt conditions. Therefore, an altered viscosity of the mucin solution cannot account for the enhanced influenza inhibition of mucin solutions at high NaCl concentrations. Instead, it is believed that multiple low-affinity bonds (based on e.g. polar and/or hydrophobic interactions) between the mucin sugar groups and the virus capsids are likely responsible for the trapping of the virus particles.

Once the detailed biochemical motifs in the mucin biopolymer are determined that are responsible for binding virus particles, then mucin biopolymers might also serve as a good role model for the de novo synthesis of engineered biopolymer materials, which trap virus particles and thus act as an artificial shielding layer towards viral infection. Indeed, a recent study employs mucin-like polysaccharide motifs in engineered polymers to trap HIV viruses in the polymer matrix. The in vitro infection assay presented herein provides a suitable platform for the screening of such engineered hydrogels to evaluate both their biocompatibility and their efficiency as an infection barrier in one convenient format.

So far, viral translocation through mucus has been studied essentially by two methods, namely single particle tracking and fluorescence recovery after photobleaching. Herein a third approach is presented, an in vitro infection assay that measures bulk translocation of virus particles through mucin solutions, or other biopolymer materials. This assay has several advantages compared to single particle tracking studies. First, it does not require fluorescence labeling of the virus capsid. Such a labelling is mandatory for optical tracking experiments with such small particles but might introduce artifacts if the label enhances or reduces adhesive interactions with the biopolymers. Second, the assay measures the bulk permeability of a biopolymer solution or a hydrogel by analyzing the infection status of a whole underlying cell layer. Thus, the infection assay directly takes into account putative heterogeneities in the hydrogel architecture, which otherwise can require numerous timeconsuming single particle measurements for their detection.

### CONCLUSION

Due to their antiviral activity, biocompatibility and availability in relatively large quantities, isolated gastric mucins are suitable candidates for supplements in personal hygiene products such as mouth rinse or tooth paste. They are also be good additives for wound treatment ointments or for genital lubricants, where they could help protecting our inner or outer body surfaces from viral infection. Mucins, such as porcine gastric mucins can be purified in bulk, and they are already used as components for artificial saliva.

TABLE
Survival of HeLa, A549 and MDCK-SIAT-1-CMV-PB1 cells after
exposure to different mucin solutions and percentage of GFP positive
cells after HPV 16 inoculation in presence of mucins.

| | | | | 1% |
|---|---|---|---|---|
| | | | 1% Sigma | NBS |
| | buffer | 1% mucin | mucin | mucin |
| Viable HeLa | ++ | ++ | ++ | −− |
| cells | | | | |
| Viable A549 | ++ | ++ | N.D. | N.D. |
| cells | | | | |
| Viable MDCK | ++ | ++ | N.D. | N.D. |
| cells | | | | |
| GFP positive | (89.5 ± 0.1)% | (3.5 ± 1.5)% | (15.5 ± 0.7)% | N.D. |
| HeLa cells | | | | |
| after HPV16 | | | | |
| inoculation | | | | |

++ := >85%.
−− := <5%.
N.D.: not determined.

It should be understood that for all numerical bounds describing some parameter in this application, such as “about,” “at least,” “less than,” and “more than,” the description also necessarily encompasses any range bounded by the recited values. Accordingly, for example, the description at least 1, 2, 3, 4, or 5 also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that it is incorporated by reference in its entirety for all purposes as well as for the proposition that is recited. Where any conflict exits between a document incorporated by reference and the present application, this application will control. All information associated with reference gene sequences disclosed in this application, such as GeneIDs, Unigene IDs, or HomoloGene ID, or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures) are hereby incorporated by reference in their entirety.

Headings used in this application are for convenience only and do not affect the interpretation of this application.

Preferred features of each of the aspects provided by the invention are applicable to all of the other aspects of the invention mutatis mutandis and, without limitation, are exemplified by the dependent claims and also encompass combinations and permutations of individual features (e.g. elements, including numerical ranges and exemplary embodiments) of particular embodiments and aspects of the invention including the working examples. For example, particular experimental parameters exemplified in the working examples can be adapted for use in the claimed invention piecemeal without departing from the invention. For example, for materials that are disclosed, while specific reference of each various individual and collective combinations and permutation of these compounds may not be explicitly disclosed, each is specifically contemplated and described herein. Thus, if a class of elements A, B, and C are disclosed as well as a class of elements D, E, and F and an example of a combination of elements, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art—thus to the extent an element is described in one or more references known to the person having ordinary skill in the art, they may be excluded from the claimed invention by, inter alia, a negative proviso or disclaimer of the feature or combination of features.

While this invention has been particularly shown and described with references to example embodiments thereof, it will be understood by those skilled in the art that various changes in form and details may be made therein without departing from the scope of the invention encompassed by the appended claims.

1. A method of inhibiting infection of one or more cells by a virus comprising contacting the one or more cells with a composition comprising biocompatible, purified, non-human gastric mucin, wherein the virus is not a Norovirus.

2. The method of claim 1 wherein the non-human gastric mucin is porcine gastric mucin.

3. The method of claim 2, wherein the porcine gastric mucin is MUC-5AC.

4-7. (canceled)

8. The method of claim 1, wherein the gastric mucin concentration is about 0.20% (w/v), 0.25% (w/v), 0.30% (w/v), 0.35% (w/v), 0.40% (w/v), 0.45% (w/v), 0.50% (w/v), 0.55% (w/v), 0.6% (w/v), 0.65% (w/v), 0.7% (w/v), 0.75% (w/v), 0.8% (w/v), 0.85% (w/v), 0.9% (w/v), 0.95% (w/v), 1% (w/v), 1.5% (w/v), 2.0% (w/v), 2.5% (w/v) when hydrated.

9. The method of claim 8, wherein the mucin concentration is between about 0.125 to about 2.0% (W/V).

10. The method of claim 9, wherein the mucin concentration is between about 0.2 to about 1.2% (W/V).

11-13. (canceled)

14. The method of claim 1, wherein the composition has a pH of about 2 to about 4.

15. The method of claim 1, wherein the composition has a salt concentration with an ionic strength equivalent to a 1:1 electrolyte of about 20 nM, 40 mM, 60 mM, 80 mM, 100 mM, 120 mM, 140 mM, 160 mM, 180 mM, 200 mM, 220 mM, 240 mM, 260 mM, 280 mM, 300 mM, 320 mM, 340 mM, 360 mM, 380 mM, 400 mM, 420 mM, 440 mM, 460 mM, 480 mM, 500 mM, 520 mM, 540 mM, 560 mM, 580 mM, or 600 mM when hydrated.

16. The method of claim 1, wherein the virus has a diameter of about 30 nm, 40 nm, 50 nm, 60 nm, 70 nm, 80 nm, 90 nm, 100 nm, 120 nm, 140 nm, 160 nm, 180 nm, or 200 nm.

17. The method of claim 1, wherein the virus is a human papilloma virus type 16 (HPV16), a Merkel cell polyoma-virus (MCV), an influenza virus, human immunodeficiency virus (HIV), Herpes simplex virus (HSV), Hepatitis B, Hepatitis C or a combination thereof.

18. The method of claim 17, wherein the virus is a human papilloma virus type 16 (HPV16), a Merkel cell polyoma-virus (MCV), an influenza virus, or a human immunodeficiency virus (HIV).

19. The method of claim 18, wherein the virus is a human papilloma virus type 16 (HPV16), a Merkel cell polyoma-virus (MCV), or an influenza A virus.

20. The method of claim 1, wherein the composition is lyophilized.

21. The method of claim 1, wherein the composition is hydrated.

22-27. (canceled)

28. The method of claim 1, wherein the composition is provided in a cream, lotion, ointment, or aerosolizable form suitable for external or internal use by a human.

29. (canceled)

30. A method of detecting translocation of one or more virus particles through a polymer comprising
a) contacting one or more cells with the one or more virus particles in the presence of the polymer, thereby producing a combination;
b) maintaining the combination under conditions which mimic the approximate time span for renewal of a mucus layer in vivo and allow translocation through the polymer; and
c) detecting whether the cells are infected with the virus particles,
wherein if the cells are infected with the virus particles, then the one or more virus particles have translocated through the polymer.

31-39. (canceled)

40. A composition comprising about 0.2-2.0% (W/V) non-recombinant mucin, about 200-500 mM NaCl or an equivalent ionic strength salt, and having a pH of about 2-4, wherein a virus exhibits a mean square displacement per second of less than 0.5 μm² on the surface.

41. The composition of claim 40, wherein the mucin is primarily porcine MUC-5AC.

42. The composition of claim 40, that is suitable for internal or external administration to a human.

43. The composition of claim 40, that is suitable for external, e.g., topical administration to a human.

44-47. (canceled)
